(19) United States
(12) Patent Application Publication
(10) Pub. No.: US 2008/0004556 A1
(43) Pub. Date: Jan. 3, 2008

(54) Multifunctional neck brace

(75) Inventors: Peter Louis Gehlbach, Monkton, MD (US); Brian Christopher Benson, Baltimore, MD (US); Helena Maria Cortes, Scarsdale, NY (US); Michael S. Davenport, Norwood, MA (US); Ryan Marques Harrison, Baltimore, MD (US); Karin Hwang, Ontario, CA (US); Gregory Michael Kapp, Gig Harbor, WA (US); Christopher Yensen Lee, Boxborough, MA (US); Joshua Adam Lerman, Delray Beach, FL (US); Tuo Li, Catonsville, MD (US); Johnson C. Wong, Little Rock, AR (US)

Correspondence Address: NIXON & VANDERHYE, PC, 901 NORTH GLEBE ROAD, 11TH FLOOR, ARLINGTON, VA, 22203, US

(73) Assignee: Johns Hopkins University, Baltimore, MD (US)

(21) Appl. No.: 11/798,041

(22) Filed: May 9, 2007

Related U.S. Application Data
(60) Provisional application No. 60/798,730, filed on May 9, 2006.

(51) Int. Cl. A61F 5/055

(52) U.S. Cl. 602/18

(57) ABSTRACT

A cervical collar that allows varying mobility to the cervical region with the range of motion or degree of mobility being adjustable and quantifiable. The customizable collar having adjustable and quantifiable mobility allows not only graduated mobility for the patient and consequently progressive strengthening but still protects the injured or diseased cervical segments. The cervical collar may also provide a user-defined and adjustable distraction force that in the medical setting allows physicians or rehabilitating specialists to adjust, graduate and monitor the amount of distraction force being applied to the cervical column. In a home use setting, patients are able to select a comfortable amount of force to counter the weight of the head. In addition to graduated rotation and/or an adjustable distraction force, a minimalist cervical collar configuration is provided that is less obtrusive, less conspicuous, light weight and can be worn in part under clothing.

This application claims the benefit of U.S. Provisional Application Ser. No. 60/798,730, which was filed May 9, 2006, the disclosure of which is incorporated herein by this reference.

### BACKGROUND OF THE INVENTION

The human spine is divided into the cervical, thoracic, lumbar and sacral vertebrae. Of these, the cervical spine (consisting of the top seven vertebrae closest to the skull) presents the highest risk of injury. This is due to a comparatively smaller amount of muscular support and the fact that they must support and bear the forces resulting from head weight and movement. The first two cervical vertebrae, the atlas (C-1) and axis (C-2) vertebrae, are designed specifically for rotation.

To understand the cause of the various types of conditions affecting the cervical spine, it is first appropriate to take a closer look at the individual bones of the spine: the vertebrae. The vertebrae are stacked one on top of the other, separated by intervertebral discs, which are soft tissues that act as shock absorbers. Between adjacent vertebrae, there is an opening called the neural foramina, through which the nerve roots exit the spine. Adjacent vertebrae also contain facet joints that prevent movements from being stiff and robotic.

Injury to the cervical spine can take many forms. One of the most common causes is whiplash, so called because it usually occurs in car accidents when the head is whipped back and forwards in a sudden fashion. Excess computer use with poor positioning of the arms and neck may also lead to the same painful symptoms of whiplash. These symptoms occur as a result of the stretching or tearing of ligaments or muscles of the neck. A sprain occurs when the ligaments are affected and a strain occurs when the muscles are affected.

Two other forms of cervical injury are known as cervical radiculopathy and cervical myelopathy. Radiculopathy is the irritation or pinching of a nerve in the neck that e.g. causes pain down to the shoulder, arm or hand. This often results from compression or mechanical irritation of the nerves as they leave the spinal cord through the neural foramen. Narrowing of the neural foraminae by a herniated disc, vertebral degeneration or bone spurs are all common contributors to disease. The surgery to correct this involves removing the disk material or spurs that are compressing the nerves and may be done in combination with fusion of some of the cervical vertebrae. Cervical myelopathy is any injury which affects the spinal cord, itself. This condition differs from cervical radiculopathy in that the injury involves the spinal cord rather than compression of exiting nerve roots.

Historically there have been many treatment approaches to relieve this common source of pain and disablement. Current best therapy is still not able to definitively address all disease. In certain cases several devices and therapies have been shown to alleviate painful symptoms of cervical injuries. However, these solutions have many shortcomings. Present day non-surgical approaches focus on decompression, or elongating the spine, in order to relieve the compression of the intervertebral discs and the pinching of nerves which result in all of the cases discussed above. Some examples of these treatments are inversion therapy and over-the-door pulley devices. While these treatments correctly address the problem of intervertebral compression via elongation and decompression of the cervical region of the spine, they lack practical features such as portability, customizability and comfort. For example, inversion therapy and pulley systems both require the patient to remain in a stationary position in order to relieve pain symptoms. For this reason, braces such as cervical collars that allow portability are often used. Simple foam braces are often used for simple neck immobilizations or as a transition from a more rigid collar to no collar. The amount of support that they provide is minimal but they are useful reminders of the positions allowed at that point in therapy. Rigid collars (such as Miami or Philadelphia types) are used when more crucial immobilizations are necessary, in emergency responses, following surgery (i.e. vertebrae fusion) in the brace does not however apply active forces to effect decompression or replace surgery. Lastly, more complex braces such as the SOMI or Halo types are used when complete immobilization of the spine is required, as may be required following complex surgery or when dangerous instability remains.

Of the above, within the continuum of cervical spine care, the Miami J® collar (by Jerome Medical) sets the standard for patient comfort and immobilization. These collars primarily consist of two rigid segments that act together to restrict neck movements and provide an immobilizing set of restrictive forces to the head and neck. While the brace itself does not relieve pressure on the cervical column a patient with an otherwise stable spine may apply a downward force to the brace that may result in some distraction force on the spine. The forces applied are not measurable with the current brace. Generally, pressure is diverted to the shoulders or the sternum. Unfortunately, this collar does not cater to the specific cervical injuries of a patient. It is medically accepted that not all patients require complete immobilization of the entire cervical column, but the current neck brace market focuses exclusively on collars that restrict motion of the entire column most efficiently. Patients who do not require complete immobilization are therefore needlessly losing usage of their upper cervical columns (specifically C-1, which allows nodding motion, and C-2, which allows the head-shaking “no” motion).

Thus, current bracing methods are attended by discomfort, lack of customizable adjustments to mobility and in fact most are completely immobilizing. Active forces generated entirely by the patient are generally diverted to the shoulders or the sternum and do not evenly distribute the weight of the head over the patient's upper body. Moreover, with a generic immobilizing design, these conventional collars are not adapted to the specific cervical injuries or disease of a patient. Indeed, known head and neck supports are generally designed to provide support in the sagittal plane, namely with the patient's head facing straight forward with zero degrees of rotation of the head and/or cervical spine. Although at times this is beneficial during the treatment of disorders including injuries, because the cervical spine is complex anatomically, biomechanically, and functionally, there are also medical conditions whose treatment would benefit by head and neck support in positions incorporating varying degrees of rotation of the cervical spine.

These effects relate to the complex movements of the anatomical cervical spine comprised of the first cervical vertebrae through the seventh cervical vertebrae. There are facet joints bilaterally between each vertebrae permitting stability through the movements of flexion, extension, side bending, and rotation. Additionally, occipital condyles at the base of the skull articulate with the first cervical vertebrae, permitting the same types of movements. There are also muscles, tendons, ligaments, and connective tissues between all of these structures, and nerve roots exit the spine on each side at each vertebrae.

When using known head and neck supports if the neck must be supported in a rotated position, these known devices must be rotated in the desired direction. Because these devices are configured for use in the sagittal plane they are not designed nor intended for use while rotated. Thus, there is a need for a head and neck support that allows the head and neck to rotate while the device does not. There is a need for a head and neck support that is able to provide support to a patient at varying, controlled, individual small ranges of head and neck rotation, without causing discomfort or imparting a tilt of the head. There is a need for a device that allows smooth rotational movement of the head within defined ranges of motion, while lower segments of the cervical column are immobilized or supported.

### BRIEF SUMMARY OF THE INVENTION

A novel neck brace for use in the decompression of the cervical region of the spinal column. Such a device will be used by patients that require chronic decompressive therapy (pre- or post-operation), patients in the perioperative period and patients with a condition that does not require surgery but are still in need of decompressive therapy for pain relief. The necessity for a novel device stems from the fact that current methods achieve cervical elongation via braces that are uncomfortable and in some cases overly immobilizing, or via even more obtrusive methods (i.e. inversion therapy and head/neck pulley systems.

The present invention provides novel neck brace assemblies for therapeutic applications including static decompression, dynamic decompression, strengthening of cervical muscles, prevention of atrophy of cervical muscles, graduating mobility and improved comfort in patents with disease attributable to the cervical region of the spinal column. The devices disclosed and suggested can be used by patients that require acute, intermittent or chronic decompression therapy. Non-operative, pre-operative and post-operative applications are envisioned. Furthermore, short term, intermittent and chronic use is proposed.

### BRIEF DESCRIPTION OF THE DRAWINGS

These, as well as other objects and advantages of this invention, will be more completely understood and appreciated by careful study of the following more detailed description of the presently preferred exemplary embodiments of the invention taken in conjunction with the accompanying drawings, in which:

FIG. 1 is a perspective view schematically illustrating a cervical collar assembly providing for mobility through selective rotation of the patient's head while supported for cervical distraction, as an embodiment of the invention;

FIG. 2 is a cross-sectional view schematically illustrating an example sliding coupling of a chin support according to an example embodiment of the invention;

FIG. 3 is a cross-sectional view schematically illustrating sliding support for the rear of the patient's head according to an example embodiment of the invention;

FIG. 4 is a cross-sectional view schematically illustrating an another example of a sliding chin support embodying the invention;

FIG. 5 is a schematic front elevation of a cervical collar assembly as another embodiment of the invention;

FIG. 6 is a schematic rear elevation of the cervical collar assembly of FIG. 5; and

FIG. 7 is a schematic plan view of the cervical collar assembly of FIG. 5.

### DETAILED DESCRIPTION OF THE INVENTION

The quality of life of the patient is always a primary concern. An object of the brace of the invention is to deliver varying mobility of the cervical region, as opposed to the pure immobility provided by conventional braces. A concern is not to immobilize regions or remove mobility that may be beneficial to the condition. Another objective of the brace is to more evenly distribute the weight of the head over the patient's upper body with intention to avoid discomfort from points of concentrated pressure.

Currently, cervical collars substantially preclude movement of a patient's neck, which is the appropriate function for many cervical injuries. But while some acute cervical injuries require complete immobilization, other patients (such as those without injuries to their C-1 and C-2 vertebrae) prefer and benefit from the ability to rotate their head from side to side. Moreover, in the case of patients recovering from cervical surgery, a collar which can be customized to suit the different time stages of recovery would also be beneficial. This is relevant since an immobilization of the spine can lead to atrophy of supporting muscles leading to further weakness and instability, whereas movement too soon after surgery can further injure the cervical segment. In short, a customizable collar would allow preservation of muscle function, gradual strengthening, and still protect the injured cervical segments.

In a first example embodiment, the present invention provides a cervical collar that allows for physician defined rotation providing for selected restricted head rotational mobility, which may be varied from less than a 35° range of motion to approximately 130°. A smooth rotational movement is allowed within the range of motion set. As mentioned above, near complete immobility is provided by current cervical braces. This serves the correct function for most cervical injury especially in the acute phases. While some acute injuries require complete immobilization, other patients, such as patients without injury or disease in their C1 and C2 vertebrae would prefer and would benefit from the ability to rotate their head from side to side which provides added function to the user during the period of brace wear such as added communication ability and added peripheral vision, important for example to driving and other aspects of daily living. Moreover, in the case of patients recovering from cervical surgery, a collar that is customized to the stage of recovery would be beneficial. Preserving mobility not only provides an immediate increase in function but serves in part avoid the atrophy and weakness that results from disease. Thus, in an example embodiment of the invention, the collar provides varying immobility to the cervical region with the range of motion or degree of mobility being adjustable and quantifiable. Indeed, the ability to adjust mobility is an important feature since too much movement too soon after surgery can further injure the cervical segment. Thus, a customizable collar would allow increased user function during brace wear, preserve muscle mass and function. A customizable collar having adjustable and quantifiable mobility allows not only graduated mobility for the patient and consequently progressive strengthening but still protects the injured or diseased cervical segments.

The invention may also be embodied in a cervical collar that provides a user-defined and adjustable distraction force that in the medical setting allows physicians or rehabilitating specialists to adjust, graduate and monitor the amount of distraction force being applied to the cervical column. In a home use setting, patients are able to select a comfortable amount of force to counter the weight of the head. The human anatomy is such that the weight of head is distributed forward, cantilever-supported by the cervical column. Furthermore, a patient suffering from compressed disks may be inclined to tuck their chin and apply a downward force onto a brace in order to create a distracting force at the cervical spine. Such a motion focuses a great deal of pressure down onto the external point of contact of the brace. A brace that more evenly distributes the weight of the head over a greater contact surface representing the area of contact of the base of the brace (back, shoulders, trapezious muscle, chest wall including sternum) alleviates the consequences of pressure points and the active forces require that the patient to create distraction. A user defined distribution system would maximize patient comfort and satisfaction.

According to yet a further embodiment, in addition to graduated rotation and/or an adjustable distraction force, a minimalist cervical collar configuration is provided that is less obtrusive, less conspicuous, light weight and can be worn in part under clothing. A minimalist model is intended to provide cervical support and allow for some distraction force to be applied, in a brace design that improves comfort and is permissive to activities of daily living. In an example embodiment, a minimalist model that is focused on decreasing the number, size and weight of components may not allow for full realization of either rotational mobility and/or adjustable distraction, nor will it fully immobilize the patient. However, patients requiring small amounts of cervical elongation and support but who need not be confined to a full cervical collar or be attached to a door pulley system, inversion therapy machine or other physical therapy/rehab techniques would be served by such a minimalist model.

The braces provided according to example embodiments of this invention distribute the weight of the head evenly to the head supporting portions of the brace and to the base of the brace. This in turn more evenly distributes the transferred force or weight of the head to the back, trapezious muscles, shoulders and chest. The distribution of forces is optimized for comfort, support and the desired therapeutic effect. These benefits are achieved by a novel design, use of superior materials and the incorporation of customizable aspects that allow the brace to be fit in a customized manner to the user.

Additional embodiments of the therapeutic neck brace embodying the invention include but are not limited to braces that allow flexion, extension, lateral tilt or rotation at a prescribed level of the brace. An example of the benefit of such a device modification is to allow a user with a normal upper cervical segment and lower cervical disease to nod the head, look down or look up.

According to a further example embodiment of the invention the brace is a rehabilitative brace for the purpose of therapeutic exercise. The rehabilitative brace provides variable resistance for all six basic neck movements in order that neck strengthening and prevention of atrophy may occur.

Various add on features may further be provided such as for example heating/cooling/ventilation/dehumidifying functions, changeable pads and attachment points for suction or other applicable devices to the care of the neck injured patient.

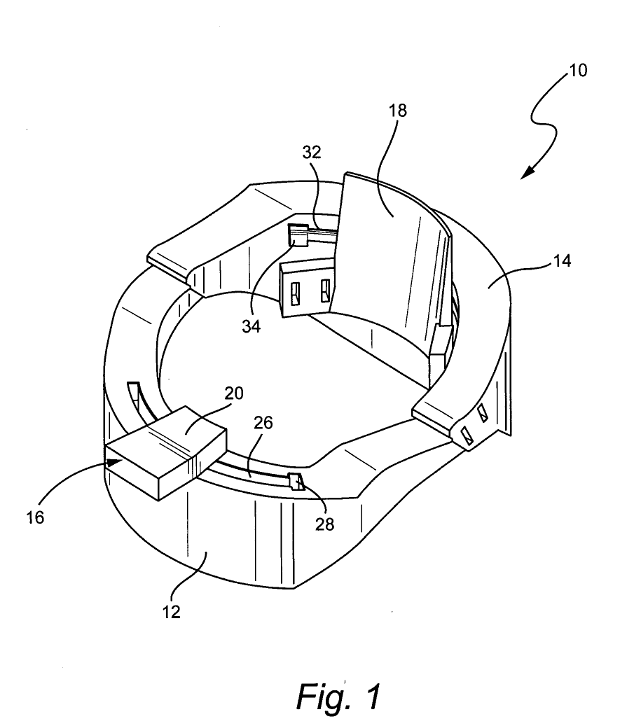

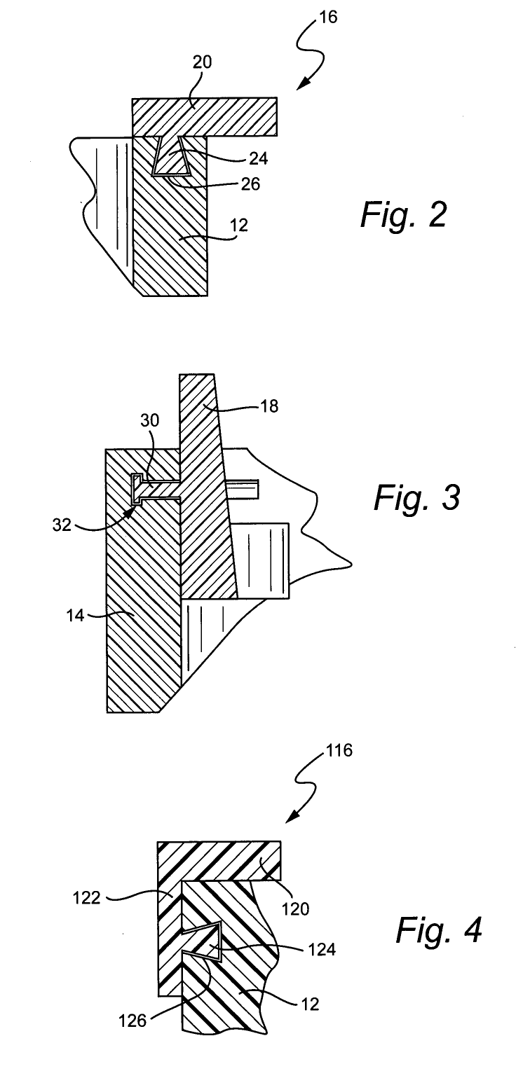

Referring to FIGS. 1-4, in a first example embodiment of the invention, a cervical collar is provided in which an adjustable and quantifiable rotary range of motion of the supported head is provided. More specifically, the invention may be embodied in a cervical collar including a chin support and preferably also a guide (which may also provide support) for the rear of the patient's head, each coupled to allow supported rotation of the patient's head relative to, the main body or frame of the cervical collar. A variety of mechanisms can be incorporated to allow for sliding movement of the chin support and/or head rest including ball bearing guide mechanisms, smooth sliding surfaces and/or smooth or lubricious materials such as Teflon and the like. Furthermore, it is to be understood that various pads and padding (not shown) would be provided in areas of contact between the patent and the brace structure for user comfort and to avoid pressure points and sores.

In the illustrated example embodiment, the cervical collar assembly 10 includes a front collar portion 12 and a rear collar portion 14 which are disposed to substantially encircle the patient's neck and transfer load respectively from the chin and rear of the patient's head to the patient's back, shoulders and chest. A fastening strap (not shown) may be coupled to the forward and/or rear components and/or provided as a separate component to encircle the collar components for securing them in position encircling the patient's neck. In the illustrated example embodiment, a chin support 16 is slidably coupled to the front collar portion and a guide 18 for the rear of the patient's head (which may be configured to support the base of the skull) is slidably coupled to the rear cervical collar portion 14.

As noted above, a variety of slide mechanisms can be used to slidably couple the chin support and the support for the base of the skull respectfully to the forward and rear components of the cervical collar. In one example embodiment, best shown in FIG. 11, the chin support 16 includes a generally horizontal component 20 which may be flat or ergonomically curved and a shaped protrusion 24 for engaging a correspondingly shaped groove 26 of the front collar portion 12. In the illustrated example embodiment the protrusion is dovetail shape, but a T-shape or other complementary protrusion/groove shape may be used. The groove 26 extends along at least a portion of the circumferential length of the forward collar portion so that the chin support 16 can be slidably displaced in an arc along and relative to a corresponding circumferential length of the front collar portion, allowing the patient whose chin is supported by the chin support 16 to correspondingly rotate his/her head. In this example embodiment, the groove 26 extends along the top of the front collar portion. In the illustrated example, furthermore, an enlarged access 28 is provided for inserting protrusion 24 into groove 26 when the chin support is coupled to the front collar portion 12.

If deemed necessary or desirable, to limit turning of the patients' head to the right or to the left or both, suitable stop(s) (not shown) can be provided on the front collar portion. For example, a stop screw or plate can be secured to a desired location along the length of the groove so that the chin support can be slid relative to the front collar portion until engagement of the protrusion 24 and/or horizontal component 20 with the set screw or stop plate. To allow adjustment while the cervical collar is worn by the patient, a plurality of pre-defined bores may be included, e.g. along the forward face of the cervical collar, and the stops, screws or the like inserted therethrough into the groove and secured as deemed appropriate. Indicia may be provided for indicating the range of motion provided by the various set screw apertures. Other ways of limiting rotation of the chin support will be readily apparent including a stop selectively secured to a front face of the front collar portion, to engage the projecting horizontal component 20 of the chin support.

As illustrated in FIGS. 1 and 3, a similar protrusion 30 and groove 32 are provided to allow the head guide 18 to be mounted to and slide relative to the rear collar portion 14. Also similar to the front of the collar, an enlarged access 34 is provided for inserting protrusion 30 into groove 32 when the head guide 18 is coupled to the rear collar portion 14. Although not illustrated, again stops may advantageously be provided to selectively limit the range of motion of the guide. Preferably, pre-formed locations for stops are correlated to those provided on the front collar portion, so that the range of motion of the head guide is correlated to that of the chin support. In this regard, the distance from the axis of rotation of the head to the head guide is less than that of the chin support so that the head support will generally slide through an arc length that is less than that of the chin support, but the angle will be the same.

In another example embodiment, as best shown in FIG. 4, the chin support 116 includes a generally horizontal component 120 which may be flat or ergonomically curved and a vertical component 122 extending downwardly from the chin support so that the chin support is generally L-shaped in cross-section. In this example, a shaped protrusion 124 for engaging a correspondingly shaped groove 126 of the front collar portion extends forwardly from the chin support vertical component 122. In this example, the protrusion is dovetail shape as in the embodiment of FIGS. 1-3, but a T-shape or other complementary protrusion/groove shape may be used. The groove 126 extends along at least a portion of the circumferential length of the forward collar portion so that the chin support 116 can be slidably displaced in an arc along and relative to the front collar portion, allowing the patient whose chin is supported by the chin support 116 to correspondingly rotate his/her head.

If it is deemed necessary or desirable to limit the turning of the patients' head to the right or to the left or both, suitable stop(s) (not shown) can be provided on the front collar portion. For example, a stop screw or plate can be secured to a desired location along the length of the groove so that the chin support can be slid relative to the front collar portion until engagement of the vertical component 122 or the protrusion 124 with the set screw or stop plate. To allow adjustment while the cervical collar is worn by the patient, a plurality of pre defined bores may be included, e.g. along the forward face of the cervical collar, and the stops, screws or the like inserted and secured as deemed appropriate therethrough into the groove 126. Indicia may be provided for indicating the range of motion provided by the various set screw apertures. Other ways of limiting rotation of the chin support will be readily apparent including a stop selectively secured to the front or upper surface of the front collar portion to engage the horizontal component 120 of the chin support, rather than the vertical component 122 or the protrusion 124.

According to a further optional feature of the invention, a user defined distraction force is incorporated in the cervical collar to allow patients or health professionals to control or adjust for comfort and therapeutic effect the amount of force to counter the weight of the head at various points around the neck. In addition to increased wearer comfort, the alleviation of symptomatic complaints such as headaches, numbness, extremity pain and others by distraction of the cervical spine indicates the presence of cervical disease and may be diagnostic as well as therapeutic.

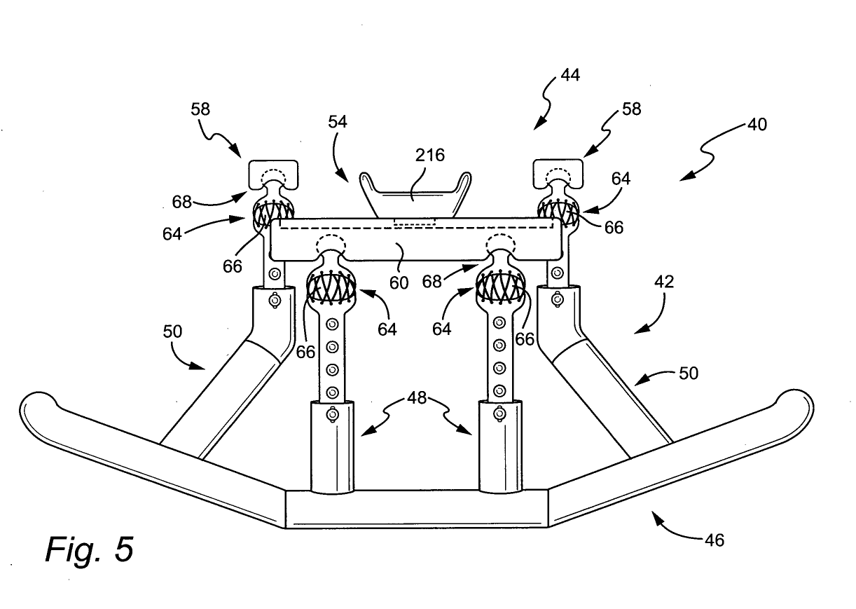

FIGS. 5-7 illustrate a further embodiment of the cervical brace or collar of the invention that incorporates the rotary feature of FIGS. 1-4, vertical adjustability for controlling distraction amount, and vertical resiliency, which may be such as to permit the patient to nod. The embodiment of FIGS. 5-7 further represents a minimalist design of the type that can be worn partly under clothing. Thus, the illustrated cervical brace 40 includes a main body 42 configured as a light weight frame for distributing the weight of the head to the patient's back, shoulders and chest. In an example embodiment, primarily for illustrative purposes, PVC tubing is used for the main body 42. It is to be understood however that a variety of materials, not limited to PVC, and a variety of component parts, not limited to tubing, can be used to construct the frame. Furthermore, it is to be understood that various pads and padding (not shown) would be provided in areas of contact between the patent and the brace structure for user comfort and to avoid pressure points and sores.

As mentioned above, main body 42 configured as a light weight frame. To that end the main body 42 is comprised of a head supporting portion 44 at and about a vertically upper periphery thereof, a weight distributing base portion 46 at and about a vertically lower periphery thereof, and a plurality of spacers 48, 50, 52 which may be static or dynamic in function extending therebetween.

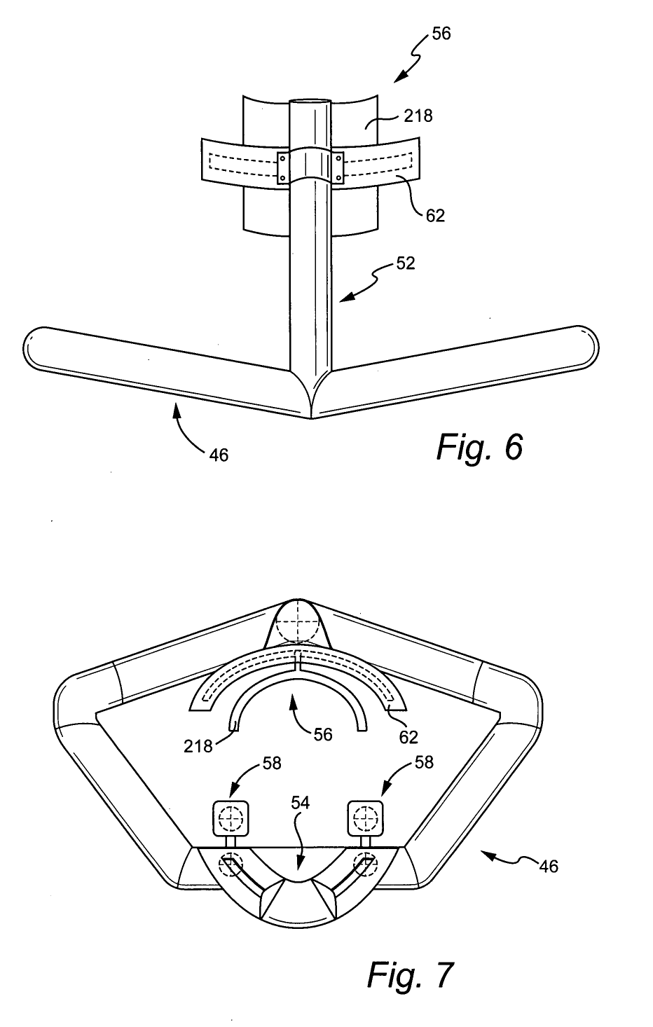

In the illustrated example, to mount the brace or collar about the patient's neck, the brace 40 is comprised of a front assembly and a rear assembly that are respectively fastened together to generally encircle and support the patient's neck. Thus, the front view of FIG. 5 is an example of the front assembly, FIG. 6 illustrates an example of the rear assembly, and FIG. 7 is a schematic top view of the attached assemblies.

To minimize the weight of the brace or collar, the head supporting portion 44 is provided as a plurality of spaced components including a chin support assembly 54, a head guide assembly 56, and, optionally, side supports 58 for engaging and supporting the sides of the jaw. The chin support assembly 54, as in the embodiments of FIGS. 1-4, may be comprised of chin support 216 slidably engaged with a groove of a front collar portion 60. Furthermore, the head guide assembly 56, as in the embodiments of FIGS. 1-4, may be comprised of guide 218 slidably engaged with a groove of a rear collar portion 62. As mentioned above, the chin support and guide are each preferably ergonomically configured and padded as deemed necessary or desirable for patient comfort.

In the illustrated embodiment, at least some of the spacers 48, 50, 52 are adjustable in length. This allows the brace to be adjusted to fit a particular patient. This also allows the patient or health professional to control or adjust, for comfort and therapeutic effect, the amount of force to counter the weight of the head at various points around the neck. In the illustrated embodiment, vertical adjustments can be effected by aligning respective holes and inserting a locking peg or the like. In the alternative, a variety of vertical adjustment mechanisms are known including ratchet and pawl mechanisms, elastic recoil as following compression of materials such as rubber, polymers or other elastic/compressible materials, shock absorber mechanisms e.g. hydraulic or similar compressible support devices that respond with a counter force that would serve to elongate the cervical spine, sliding locking nut, turnbuckle, and/or other continuous slide mechanisms as, for example disclosed in U.S. Pat. Nos. 6,726,643, 4,099,523; 5,00,563; 6,423,020, and 5,865,773, the disclosures of each of which are incorporated herein by these references. Thus, the height adjustment can be incremental, e.g. with a plurality of spaced coupling components, such as the illustrated pin and bore arrangement; a nearly continuous adjustment, as with a ratchet and pawl mechanism; or a substantially continuous adjustment, as with a sliding locking nut or with a turnbuckle or other threaded adjustment mechanism.

Because the bulk of the weight of the patients' head is borne at the front, in the illustrated example embodiment four spacers or vertical supports 48, 50 are defined to extend between the weight distributing base portion 46 and the chin support assembly 54. In the proposed embodiment, each of the front spacers or vertical supports 48, 50 is configured to be selectively vertically adjusted, as described above, to provide the weight support and distraction appropriate to the particular patient. Furthermore, in this embodiment, each of the front spacers 48, 50 includes a resilient segment 64 for resiliently supporting the patients' head, to allow the patient to nod to a limited extent while compressing the resilient segment. In the illustrated example embodiment an elastomeric (rubber) ball 66 is provided as resilient segment 64. It is to be understood, however, that a variety of other resilient support segments could be inter-changed for the rubber ball. For example a spring, mini hydraulic cylinder, viscous putty, or other resiliently compressible component could be provided in its stead. In the illustrated embodiment, universal joints 68 are optionally defined between the upper end of the spacers 48, 50 and the head supporting component(s), more specifically the front collar portion 60, (and the optional side supports 58) to accommodate nodding articulation of the head.

In the illustrated example embodiment, the rear head guide assembly 56 is not vertically adjustable, but it is to be understood that an adjustable spacer, with or without resilient segment, could be provided therefor.

The invention in the example embodiments incorporates vertical adjustment while retaining resiliency, preferably a resilient component associated with each adjustable vertical support. This resilient distraction adjustment can be provided alone or together with accommodation for rotation of the head. Preferably, one or the other or both of these capabilities are incorporated in a minimalist frame for a wide distribution of the supported weight.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A cervical collar assembly comprising:
a main body configured to substantially encircle a patient's neck and including a front collar portion and a rear collar portion adapted to respectively transfer load from the chin and rear of the patient's head to the patient's back, shoulders and chest;
a chin support slidably coupled to the front collar portion; and
a guide for the rear of the patient's head coupled to the rear collar portion.

2. A cervical collar assembly as in claim 1, wherein said chin support is slidably coupled so as to be slidable through an arc of between about 35° and about 130°.

3. A cervical collar assembly as in claim 1, wherein said chin support includes a generally horizontal component and a shaped protrusion for engaging a correspondingly shaped groove in the front collar portion.

4. A cervical collar assembly as in claim 3, wherein said protrusion is dovetail shaped for engaging a complimentary shaped groove.

5. A cervical collar assembly as in claim 3, wherein the groove extends along at least a portion of the circumferential length of the forward collar portion so that the chin support can be slidably displaced in an arc along and relative to a corresponding circumferential length of the front collar portion.

6. A cervical collar assembly as in claim 3, wherein the groove extends along the top of the front collar portion.

7. A cervical collar assembly as in claim 5, wherein said groove is defined so as to define a range of motion for the patient's head of between about 35° and about 130°.

8. A cervical collar assembly as in claim 3, wherein said groove includes an enlarged access aperture for inserting the protrusion into the groove.

9. A cervical collar assembly as in claim 1, wherein said the chin support includes a generally horizontal component and a vertical component so that the chin support is generally L-shaped in cross-section and includes a protrusion for engaging a correspondingly shaped groove of the front collar portion.

10. A cervical collar assembly as in claim 1, wherein said guide is slidably coupled to the rear collar portion.

11. A cervical collar assembly as in claim 10, wherein the head guide includes a protrusion disposed in a correspondingly shaped groove of the rear collar portion.

12. A minimalist cervical collar assembly comprising:
a main body configured as a light weight frame including a head supporting portion at and about a vertically upper periphery thereof, a weight distributing base portion at and about a vertically lower periphery thereof, and a plurality of vertical supports extending therebetween;
wherein the head supporting portion includes a chin support assembly and a head guide assembly;
wherein at least some of the vertical supports are adjustable in length.

13. A minimalist cervical collar assembly as in claim 12, wherein the main body is comprised of a front assembly and a rear assembly that are respectively fastened together to generally encircle the patient's neck and support the patient's head.

14. A minimalist cervical collar assembly as in claim 12, wherein a plurality of said vertical supports are provided as front vertical supports to extend between the weight distributing base portion and the chin support assembly.

15. A minimalist cervical collar assembly as in claim 14, wherein each of said front vertical supports includes a resilient segment for resiliently supporting the patient's head whereby the patient can nod to a limited extent while compressing the resilient segments.

16. A minimalist cervical collar assembly as in claim 15, wherein at least one said resilient segment comprises an elastomeric material.

17. A minimalist cervical collar assembly as in claim 12, wherein universal joints are defined between upper ends of the vertical supports and the head supporting portion.

18. A minimalist cervical collar assembly as in claim 12, wherein the head supporting portion further comprises side supports for engaging and supporting the sides of the patient's jaw.

19. A minimalist cervical collar assembly as in claim 12, wherein said chin support assembly comprises a chin support slidably engaged with a front collar portion of the head supporting portion.

20. A minimalist cervical collar assembly as in claim 12, wherein said head guide assembly comprises a head guide slidably engaged with a rear collar portion of the head supporting portion.
